(19) United States
(12) Patent Application Publication
(10) Pub. No.: US 2011/0190306 A1
(43) Pub. Date: Aug. 4, 2011

(54) Inhibitors of PLK

(75) Inventors: David Festus Charles Moffat, Oxfordshire (GB); Sanjay Ratilal Patel, Oxfordshire (GB); Kenneth William John Baker, Oxfordshire (GB); Carl Leslie North, Oxfordshire (GB)

(73) Assignee: CHROMA THERAPEUTICS LTD., ABINGDON, OXFORDSHIRE (GB)

(21) Appl. No.: 12/989,178

(22) PCT Filed: Apr. 23, 2009
(86) PCT No.: PCT/GB09/01019

§ 371 Date: Nov. 12, 2010

(30) Foreign Application Priority Data
Apr. 24, 2008 (GB) 0807451.0

(51) Int. Cl. A61K 31/519; C07D 475/00; A61P 35/00

(52) U.S. Cl. 514/249; 544/258

(57) ABSTRACT

Compounds of formula (I) are PLK inhibitors, useful for the treatment of cell proliferative diseases: wherein R₁ is hydrogen, or an optionally substituted (C₁-C₆)alkyl, (C₂-C₆)alkenyl, (C₂-C₆)alkynyl or (C₃-C₆)cycloalkyl group; R₂ is hydrogen, or an optionally substituted (C₁-C₆)alkyl, (C₂-C₆)alkenyl, (C₂-C₆)alkynyl or (C₃-C₆)cycloalkyl group; R₃ is hydrogen, —CN, hydroxyl, halogen, optionally substituted (C₁C₆)alkyl, (C₂-C₆)alkenyl, (C₂-C₆)alkynyl or (C₃-C₆)cycloalkyl, —NR₅R₆ or C₁-C₄ alkoxy, wherein R₅ and R₆ are independently hydrogen or optionally substituted (C₁-C₆)alkyl; ring A is an optionally substituted mono- or bi-cyclic carbocyclic or heterocyclic ring or a ring system having up to 12 ring atoms; T is a radical of formula R-L¹-Y¹— wherein L¹ and Y¹ are as defined in the claims and R is an carbon-linked, alpha alpha disubstituted amino acid or amino acid ester residue.

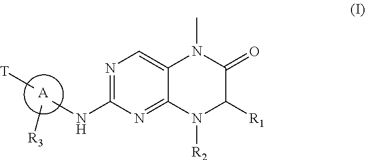

This invention relates to a series of amino acid esters, to compositions containing them, to processes for their preparation and to their use in medicine as Polo-like kinase ‘PLK’ inhibitors. Polo-like kinases (PLKs) are key enzymes that control mitotic entry of proliferating cells and regulate many aspects of mitosis necessary for successful cytokinesis. Of the four known human PLKs, PLK1 is the best characterized and is overexpressed in many tumour types with aberrant elevation frequently constituting a prognostic indicator of poor disease outcome. The compounds may be of use in the treatment of cell proliferative diseases such as cancer. The present invention encompasses compounds that are dihydropteridinine derivatives.

### BACKGROUND TO INVENTION

The PLKs, a family of Ser/Thr protein kinases named after their functional and sequence similarity with the archetypal polo kinase from Drosophila melanogaster, play a variety of roles in mitosis (Nat. Rev. Mol. Cell Biol., 2001, 2, 21-32). In yeasts (Saccharomyces cerevisiae and S. pombe) single PLKs exist, whereas four distinct PLKs have been identified to date in mammals. Human PLK1 (Cell Growth Differ., 1994, 5, 249-257), PLK2 (serum-inducible kinase, SNK, Mol. Cell. Biol., 1992, 12, 4164-4169), PLK3 (proliferation-related kinase, PRK J. Biol. Chem. 1997, 272, 28646-28651) and PLK4 (Oncol. Rep., 1997, 4, 505-510) are structurally homologous and contain two conserved domains, the N-terminal catalytic kinase domain, as well as a C-terminal region composed of the so-called polo boxes. Whereas PLK1, PLK2, and PLK3 are expressed in all tissues, PLK4 appears to possess unique physiological roles and the distribution of PLK4 mRNA in adults is restricted to certain tissues such as testes and thymus. PLK1 is the best characterized member of the PLK family and it appears to fulfil most of the known functions of the single PLKs present in invertebrates (Nat. Rev. Mol. Cell. Biol., 2004, 5, 429-441). PLK1 protein levels fluctuate in a cell-cycle-dependent manner and its kinase activity peaks at the transition between the second gap phase and the mitosis phases (G2/M) of the eukaryotic cell division cycle. Upon exit from mitosis PLK1 levels drop as a result of ubiquitin-dependent proteolysis. PLK1 has been reported to be involved in the initiation of mitosis through activation of the cyclin-dependent kinase CDK1/cyclin B complex, i.e. the master switch for mitotic entry (mitosis-promoting factor, MPF Nature, 1990, 344, 503-508).

This occurs when PLK1 phosphorylates, and thus activates, the dual specificity phosphatase CDC25C, which in turn relieves premitotic MYT1- and WEE1-mediated suppression of CDK1/cyclin B activity through dephosphorylation at the CDK1 pThr14 and pTyr15 sites (Cell, 1991, 67, 197-211). Upon entry into mitosis, phosphorylation of CDC25C by PLK1 and PLK3 leads to its translocation into the nucleus. Apart from controlling entry into mitosis through CDK1 activation, PLK1 has additional roles in regulating progression through mitosis. It is involved in bipolar spindle formation, including centrosome maturation and regulation of the microtubule organizing centre, in the subsequent steps of mitosis involving sister chromatid separation, and finally in cytokinesis (Dev. Cell, 2003, 5, 127-138).

### BRIEF SUMMARY OF THE INVENTION

Compounds of the invention are related to compounds disclosed in WO2004076454. They are inhibitors of PLK1 and the isoforms thereof. The compounds are thus of use in medicine, for example in the treatment of a variety of proliferative disease states, including cancers. The compounds are characterised by the presence in the molecule of an α,α-disubstituted glycine acid motif or an α,α-disubstituted glycine ester motif which is hydrolysable by an intracellular carboxylesterase. Compounds of the invention having the lipophilic α,α-disubstituted glycine ester motif cross the cell membrane, and are hydrolysed to the acid by the intracellular carboxylesterases. The polar hydrolysis product accumulates in the cell since it does not readily cross the cell membrane. Hence the PLK1 activity of the compound is prolonged and enhanced within the cell.

### DETAILED DESCRIPTION OF THE INVENTION

According to the invention there is provided a compound of formula (I), or a salt thereof:

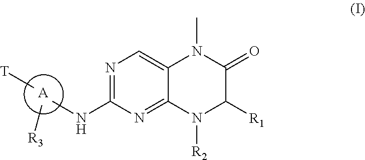

wherein
R₁ is hydrogen, or an optionally substituted (C₁-C₆)alkyl, (C₂-C₆)alkenyl, (C₂-C₆)alkynyl or (C₃-C₆)cycloalkyl group;
R₂ is hydrogen, or an optionally substituted (C₁-C₆)alkyl, (C₂-C₆)alkenyl, (C₂-C₆)alkynyl or (C₃-C₆)cycloalkyl group;
R₃ is hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₅R₆ or (C₁-C₄)alkoxy, wherein R₅ and R₆ are independently hydrogen or optionally substituted (C₁-C₆)alkyl;
ring A is an optionally substituted mono- or bi-cyclic carbocyclic or heterocyclic ring or a ring system having up to 12 ring atoms;
T is a radical of formula R-L¹-Y¹— wherein
Y¹ is a bond, —O—, —S—, —NR₆—, —(C═O)—, —S(O₂)—, —(C═O)NR₆—, —NR₆(C═O)—, —S(O₂)NR₆—, —NR₆S(O₂)—, or —NR₆(C═O)NR₉—, wherein R₆ and R₉ are independently hydrogen or optionally substituted (C₁-C₆)alkyl;
L¹ is a divalent radical of formula -(Alk¹)ₘ(Q)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl;
   R is a radical of formula (X)

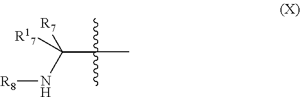

wherein
R₇ is a carboxylic acid group (—COOH), or an ester group which is hydrolysable by one or more intracellular carboxylesterase enzymes to a carboxylic acid group;
R¹₇ is the side chain of a natural or non-natural alpha-amino acid, in which any functional groups are protected, but R¹₇ is not hydrogen;
R₈ is hydrogen; or optionally substituted C₁-C₆ alkyl, C₃-C₇ cycloalkyl, aryl or heteroaryl or —(C═O)R₆, —(C═O)OR₆, or —(C═O)NR₆ wherein R₆ is hydrogen or optionally substituted (C₁-C₆)alkyl.

In the compounds of the invention, when R₁ is other than hydrogen, the carbon atom to which the R₁ substituent is attached is asymmetric. Preferably the stereo chemistry at that asymmetric center is R.

In another broad aspect the invention provides the use of a compound of formula (I) as defined above, or an N-oxide, salt, hydrate or solvate thereof in the preparation of a composition for inhibiting the activity of PLK1.

The compounds with which the invention is concerned may be used for the inhibition of PLK1 activity ex vivo or in vivo.

In one aspect of the invention, the compounds of the invention may be used in the preparation of a composition for treatment of cell proliferative diseases such as cancer.

In another aspect, the invention provides a method for the treatment of the foregoing disease types, which comprises administering to a subject suffering such disease an effective amount of a compound of formula (I) as defined above.

### Terminology

As used herein, the term “(Cₐ-C_{b})alkyl” wherein a and b are integers, refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein, the term “divalent (Cₐ-C_{b})alkylene radical”, wherein a and b are integers, refers to a saturated hydrocarbon chain having from a to b carbon atoms and two unsatisfied valences.

As used herein, the term “(Cₐ-C_{b})alkenyl” wherein a and b are integers, refers to a straight or branched chain alkenyl moiety with a to b carbon atoms; having at least one double bond of either E or Z stereochemistry where applicable. The term includes, for example, vinyl, allyl, 1- and 2-butenyl and 2-methyl-2-propenyl.

As used herein, the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl”, wherein a and b are integers refers to straight chain or branched chain hydrocarbon groups having from two to six carbon atoms and having in addition one triple bond. This term would include, for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein, the term “divalent (Cₐ-C_{b})alkynylene radical”, wherein a and b are integers refers to a divalent hydrocarbon chain having from two to six carbon atoms, and at least one triple bond.

As used herein, the term “carbocyclic” refers to a mono-, bi- or tricyclic radical having up to 16 ring atoms, all of which are carbon, and includes aryl and cycloalkyl.

As used herein, the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein, the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein, the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein, the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

A “divalent phenylene, pyridinylene, pyrimidinylene, or pyrazinylene radical” is a benzene, pyridine, pyrimidine or pyrazine ring, with two unsatisfied valencies, and includes 1,3-phenylene, 1,4-phenylene, and the following:

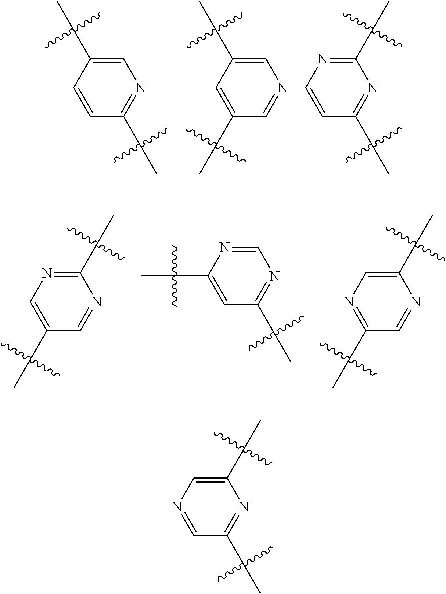

Unless otherwise specified in the context in which it occurs, the term “substituted”, as applied to any moiety herein, means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl or monocyclic heteroaryl having 5 or 6 ring atoms, or R^{A} and R^{B} when attached to the same nitrogen atom form a cyclic amino group (for example morpholino, piperidinyl, piperazinyl, or tetrahydropyrrolyl). An “optional substituent” may be one of the foregoing substituent groups.

The term “side chain of a natural or non-natural alpha-amino acid” refers to the group R^{Y} in a natural or non-natural amino acid of formula NH₂—CH(R^{Y})—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, α-amino-n-butyric acid, 3,4-dihydroxyphenylalanine, homoserine, α-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R¹₇ in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulphonic, glutamic, lactic, and mandelic acids and the like.

It is expected that compounds of the invention may be recovered in N-oxide, hydrate or solvate form, and such forms are expected to have the activity of the non-hydrated, non-solvated or non-N-oxidised forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds of the invention which contain one or more actual or potential chiral centres, because of the presence of asymmetric carbon atoms, can exist as a number of diastereoisomers with R or S stereochemistry at each chiral centre. The invention includes all such diastereoisomers and mixtures thereof.

The term “ester” or “esterified carboxyl group” in connection with substituent R₇ above means a group R₁₀O(C═O)— in which R₁₀ is the group characterising the ester, notionally derived from the alcohol R₁₀OH.

### The Substituents R₁-R₃

R₁ is hydrogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl. In one subclass of compounds of the invention R₁ is ethyl.

R₂ is hydrogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl, or C₆₋₁₄ aryl for example phenyl or naphthyl. In one subclass of compounds of the invention R₂ is cyclopentyl.

R₃ is hydrogen, —CN, hydroxyl, halogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl, —NR₅R₆ and C₁-C₄ alkoxy, wherein R₅ and R₆ are independently hydrogen or optionally substituted (C₁-C₆)alkyl, for example methyl or ethyl. In one subclass of compounds of the invention R₃ is hydrogen.

### The Ring A

Ring A is a mono- or bi-cyclic carbocyclic or heterocyclic ring or a ring system having up to 12 ring atoms. Examples of such rings are piperidine, piperazine, pyridine, pyrimidine, pyrazoline, triazoline, furan, thiophene, pyrrole, thiazole, isothiazole, oxazole, isoxazole, and thiadiazole rings. Currently preferred rings A are phenyl, pyridinyl and pyrimidinyl.

Ring A may be substituted by any of the optional substituents referred to above, for example chloro, bromo or fluoro, trifluoromethyl, methoxy, and trifluoromethoxy.

### The Substituent T

This substituent contains the α,α-disubstituted glycine acid or α,α-disubstituted glycine ester moiety of formula (X) linked through a linker radical to ring A.

The ester compounds of the invention are converted by intracellular esterases to the carboxylic acid. Both the esters and carboxylic acids may have PLK inhibitory activity in their own right. The compounds of the invention therefore include not only the ester, but also the corresponding carboxylic acid hydrolysis products.

### The Ester Group R₇

The ester group R₇ present in substituent T must be one which in the compound of the invention is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes other enzymes such as biphenylhydrolase (BPH) may also have a role in hydrolysing the conjugates. In general, if the carboxylesterase hydrolyses the free amino acid ester to the parent acid it will also hydrolyse the ester motif when covalently conjugated to the modulator. Hence, the broken cell assay described herein provides a straightforward, quick and simple first screen for esters which have the required hydrolysis profile. Ester motifs selected in that way may then be re-assayed in the same carboxylesterase assay when conjugated to the rest of the molecule via the chosen conjugation chemistry, to confirm that it is still a carboxylesterase substrate in that background.

Subject to the requirement that they be hydrolysable by intracellular carboxylesterase enzymes, examples of particular ester groups R₇ include those of formula —(C═O)OR₁₀ wherein R₁₀ is R₁₁R₁₂R₁₃C— wherein
(i) R₁₁ is hydrogen, fluorine or optionally substituted (C₁-C₃)alkyl-(Z¹)ₐ—[(C₁-C₃)alkyl]_{b}— or (C₂-C₃)alkenyl-(Z¹)ₐ—[(C₁-C₃)alkyl]_{b}— wherein a and b are independently 0 or 1 and Z¹ is —O—, —S—, or —NR₁₄— wherein R₁₄ is hydrogen or (C₁-C₃)alkyl; and R₁₂ and R₁₃ are independently hydrogen or (C₁-C₃)alkyl-;
(ii) R₁₁ is hydrogen or optionally substituted R₁₅R₁₆N—(C₁-C₃)alkyl- wherein R₁₅ is hydrogen or (C₁-C₃)alkyl and R₁₆ is hydrogen or (C₁-C₃)alkyl; or R₁₅ and R₁₆ together with the nitrogen to which they are attached form an optionally substituted monocyclic heterocyclic ring of 5- or 6-ring atoms or bicyclic heterocyclic ring system of 8 to 10 ring atoms, and R₁₂ and R₁₃ are independently hydrogen or (C₁-C₃)alkyl-; or
(iii) R₁₁ and R₁₂ taken together with the carbon to which they are attached form an optionally substituted monocyclic carbocyclic ring of from 3 to 7 ring atoms or bicyclic carbocyclic ring system of 8 to 10 ring atoms, and R₁₃ is hydrogen.

In cases (i), (ii) and (iii) above, “alkyl” includes fluoroalkyl.

Within these classes, R₁₀ may be, for example, methyl, trifluoromethyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl, or morpholinoethyl. Currently preferred is where R₁₀ is cyclopentyl.

### The Group R¹₇

The group R¹₇ present in substituent T is the side chain of a natural or non-natural alpha-amino acid, in which any functional groups are protected, but R¹₇ is not hydrogen.

For example, R¹₇ may be phenyl, or heteroaryl such as pyridyl, or a group of formula —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-Cₛ)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

In some cases, R¹₇ is H-Alk⁴-, phenyl, monocyclic heterocyclyl, C₃-C₇ cycloalkyl, phenyl(Alk⁴)-, heterocyclyl(Alk⁴)-, or C₃-C₇ cycloalkyl(Alk⁴)-, wherein the heterocyclyl part is monocyclic heterocyclyl having 3-7 ring atoms, and wherein -Alk⁴- is a straight or branched, divalent (C₁-C₆)alkylene, (C₂-C₆)alkenylene, or (C₂-C₆)alkynylene radical which may optionally be interrupted by, or terminate in, an ether (—O—), thioether (—S—) or amino (—NR^{A}—) link wherein R^{A} is hydrogen or optionally substituted (C₁-C₃)alkyl, and wherein the Alk⁴-, or cyclic part is optionally substituted. For example, R¹₇ may be C₁-C₆ alkyl substituent, for example methyl, ethyl, n- or iso-propyl, or n-, sec- or tert-butyl.

In a particular case, R¹₇ is methyl.

### The Group R₈

R₈ may be, for example, optionally substituted (C₁-C₆)alkyl, (C₃-C₆)cycloalkyl, aryl or heteroaryl, for example methyl, ethyl, n- or isopropyl, cyclopropyl, cyclopentyl, cyclohexyl, phenyl, or pyridyl. R₈ may also be, for example hydrogen or —(C═O)R₁₆, wherein R₁₆ is optionally substituted (C₁-C₆)alkyl such as methyl, ethyl, n- or isopropyl, or n-, iso- or sec-butyl, (C₃-C₆)cycloalkyl such as cyclopropyl, cyclopentyl, cyclohexyl, phenyl, pyridyl, thienyl, phenyl(C₁-C₆alkyl)-, thienyl(C₁-C₆alkyl)- or pyridyl(C₁-C₆alkyl)- such as benzyl, 4-methoxyphenylmethylcarbonyl, thienylmethyl or pyridylmethyl.

R₈ may also be, for example —(C═O)OR₁₇, or —(C═O)NHR₁₇ wherein R₁₇ is hydrogen or optionally substituted (C₁-C₆)alkyl such as methyl, ethyl, or n- or isopropyl.

Currently it is preferred that R₈ be hydrogen.

For compounds of the invention which are to be administered systemically, esters with a slow rate of esterase cleavage are preferred, since they are less susceptible to pre-systemic metabolism. Their ability to reach their target tissue intact is therefore increased, and the ester can be converted inside the cells of the target tissue into the acid product. However, for local administration, where the ester is either directly applied to the target tissue or directed there by, for example, inhalation, it will often be desirable that the ester has a rapid rate of esterase cleavage, to minimise systemic exposure and consequent unwanted side effects. If a carbon atom to which the group R is attached is unsubstituted, ie R is attached to a methylene (—CH₂)— radical, then the esters tend to be cleaved more rapidly than if that carbon is substituted, or is part of a ring system such as a phenyl or cyclohexyl ring.

### The Radical -L¹-Y¹—

This radical (or bond) arises from the particular chemistry strategy chosen to link the amino acid ester motif R in substituent T to ring A of the inhibitor. Clearly the chemistry strategy for that coupling may vary widely, and thus many combinations of the variables Y¹ and L¹ are possible. However, when the inhibitor is bound to the enzyme at its active site, the amino acid ester motif generally extends in a direction away from the enzyme, and thus minimises or avoids interference with the binding mode of the inhibitor. Hence the precise combination of variable making up the linking chemistry between the amino acid ester motif and the rest of the molecule will often be irrelevant to the primary binding mode of the compound as a whole.

With the foregoing general observations in mind, taking the variables making up the radical -L¹-Y¹— in turn:
Y¹ may be, for example, —NR₃—, —S—, —O—, —C(═O)NR₃—, —NR₃C(═O)—, or —C(═O)O—, wherein R₃ is hydrogen or optionally substituted C₁-C₆ alkyl such as —CH₂CH₂OH;
In the radical L¹, examples of Alk¹ and Alk² radicals, when present, include —CH₂—, —CH₂CH₂—, —CH₂CH₂CH₂—, —CH₂CH₂CH₂CH₂—, —CH═CH—, —CH═CHCH₂—, —CH₂CH═CH—, CH₂CH═CHCH₂—, —C≡C—, —C≡CCH₂—, —CH₂C≡C—, and CH₂C≡CCH₂. Additional examples of Alk¹ and Alk² include —CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals.
Alk¹ and Alk² when present may also be branched chain alkyl such as —CH(CH₃)—, —C(CH₃)₂—, or in either orientation —CH₂CH(CH₃)—, —CH₂C(CH₃)₂—.
In L¹, when n is 0, the radical is a hydrocarbon chain (optionally substituted and perhaps having an ether, thioether or amino linkage). Presently it is preferred that there be no optional substituents in L¹. When both m and p are 0, L¹ is a divalent mono- or bicyclic carbocyclic or heterocyclic radical with 5-13 ring atoms (optionally substituted). When n is 1 and at least one of m and p is 1, L¹ is a divalent radical including a hydrocarbon chain or chains and a mono- or bicyclic carbocyclic or heterocyclic radical with 5-13 ring atoms (optionally substituted). When present, Q may be, for example, a divalent phenyl, naphthyl, cyclopropyl, cyclopentyl, or cyclohexyl radical, or a mono-, or bi-cyclic heterocyclic radical having 5 to 13 ring members, such as piperidinyl, piperazinyl, indolyl, pyridyl, thienyl, or pyrrolyl radical, but 1,4-phenylene is presently preferred.
Specifically, in some embodiments of the invention, 12, m and p may be 0 with n being 1. In other embodiments, n and p may be 0 with m being 1. In further embodiments, m, n and p may be all 0. In still further embodiments m may be 0, n may be 1 with Q being a monocyclic heterocyclic radical, and p may be 0 or 1. Alk¹ and Alk², when present, may be selected from —CH₂—, —CH₂CH₂—, and —CH₂CH₂CH₂— and Q may be 1,4-phenylene.

Specific examples of the radical -L¹-Y¹—[CH₂]_{z}— include —(CH₂)₃NH—, —CH₂C(═O)NH—, —CH₂CH₂C(═O)NH—, —CH₂C(O)O—, —CH₂S—, —CH₂CH₂C(O)O—, —(CH₂)₄NH—, —CH₂CH₂S—, —CH₂O, —CH₂CH₂O—,

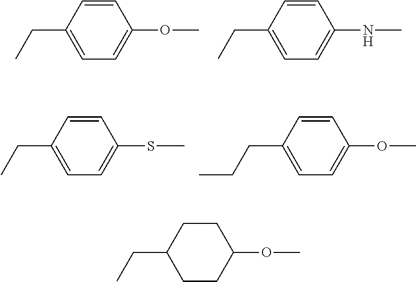

In a specific preferred example, useful when ring A is a phenyl ring, of the radical -L¹-Y¹—, L¹ is C₁-C₃ alkylene, eg —CH₂—, —CH₂CH₂— or —CH₂CH₂CH₂—, and Y¹ is —NHC(═O)—.

As mentioned above, the compounds with which the invention is concerned are inhibitors of PLK1 kinase activity and are therefore of use for treatment of cell proliferative diseases such as cancer.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavourings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds of the invention may be used in conjunction with a number of known pharmaceutically active substances. For example, the compounds of the invention may be used with cytotoxics, HDAC inhibitors, kinase inhibitors, aminopeptidase inhibitors, protease inhibitors, bcl-2 antagonists, inhibitors of mTor and monoclonal antibodies (for example those directed at growth factor receptors). Preferred cytotoxics include, for example, taxanes, platins, anti-metabolites such as 5-fluoracil, topoisomerase inhibitors and the like. The medicaments of the invention comprising amino acid derivatives of formula (I), tautomers thereof or pharmaceutically acceptable salts, N-oxides, hydrates or solvates thereof therefore typically further comprise a cytotoxic, an HDAC inhibitor, a kinase inhibitor, an aminopeptidase inhibitor and/or a monoclonal antibody.

Further, the present invention provides a pharmaceutical composition comprising:
(a) a compound (I), or a pharmaceutically acceptable salt, N-oxide, hydrate or solvate thereof;
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody; and
(c) a pharmaceutically acceptable carrier or diluent.

Also provided is a product comprising:
(a) a compound (I), or a pharmaceutically acceptable salt, N-oxide, hydrate or solvate thereof; and
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody,
   for the separate, simultaneous or sequential use in the treatment of the human or animal body.

### Synthesis

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to those skilled in the art. Typical literature sources are “Advanced organic chemistry”, 4^{th} Edition (Wiley), J March; “Comprehensive Organic Transformation”, 2^{nd} Edition (Wiley), R. C. Larock, “Handbook of Heterocyclic Chemistry”, 2^{nd} Edition (Pergamon), A. R. Katritzky; review articles such as found in “Synthesis”, “Acc. Chem. Res.”, “Chem. Rev.”, or primary literature sources identified by standard literature searches online or from secondary sources such as “Chemical Abstracts” or “Beilstein”.

The compounds of the invention may be prepared by a number of processes some of which are described specifically in the Examples below. In the reactions described below, it may be necessary to protect reactive functional groups, for example hydroxyl, amino and carboxy groups, where these are desired in the final product, to avoid their unwanted participation in the reactions [see for example, “Protecting Groups in Organic Synthesis”, 3^{rd} Edition, (Wiley), T. W. Greene]. Conventional protecting groups may be used in conjunction with standard practice. In some instances deprotection may be the final step in the synthesis of a compound of general formula (I), and the processes according to the invention described herein after are understood to extend to such removal of protecting groups.

### Abbreviations

AcOH=acetic acid
EDC=1-ethyl-3-(3-dimethylaminopropyl)carbodiimide
EtOAc=ethyl acetate
EtOH=ethanol
Et₂O=diethyl ether
Et₃N=triethylamine
Boc or boc=tert-butoxycarbonyl

### Boc₂O=Di-tert-butyldicarbonate

Cbz=benzyloxycarbonyl
DCE=dichloroethane
DCM=dichloromethane
DIPEA=diisopropylethylamine
DMAP=dimethylamino pyridine
DMF=dimethylformamide
DMSO=dimethyl sulfoxide
HCl=hydrochloric acid
K₂CO₃=potassium carbonate
LiOH=lithium hydroxide
MeOH=methanol
MgSO₄=magnesium sulphate
Na₂CO₃=sodium carbonate
NaH=sodium hydride
NaHCO₃=sodium hydrogen carbonate
NaI=sodium iodide
NaOH=sodium hydroxide
NBu₄Br=tetrabutylammonium bromide
Pd(dppf)Cl₂=dichloro-(1,2-bis-(diphenylphosphino)ethane)-palladium(II)
Pd/C=palladium on carbon
STAB=sodium triacetoxyborohydride
TBTU=O-benzotriazol-1-yl-N,N,N′,N′-tetramethyluronium tetrafluoroborate
TFA=trifluoroacetic acid
THF=tetrahydrofuran
aq=aqueous
g=gram(s)
LCMS=high performance liquid chromatography/mass spectrometry
mg=milligram(s)
min=minutes
mL=milliliter(s)=
microlitre(s)
mol=mole(s)
mmol=millimole(s)
NMR=nuclear magnetic resonance
RT or rt=room temperature
sat=saturated

Commercially available reagents and solvents (HPLC grade) were used without further purification. Solvents were removed using a Buchi rotary evaporator. Microwave irradiation was carried out using a Biotage Initiator™ Eight microwave synthesiser. Purification of compounds by flash chromatography column was performed using silica gel, particle size 40-63 μm (230-400 mesh) obtained from Fluorochem. Purification of compounds by preparative HPLC was performed on Gilson systems using reverse phase Axia™ prep Luna C18 columns (10 μm, 100×21.2 mm), gradient 0-100% B (A=water/0.05% TFA, B=acetonitrile) over 10 min, flow=25 mL/min, UV detection at 254 nm.

¹H NMR spectra were recorded on a Bruker 300 MHz AV spectrometer in deuterated solvents. Chemical shifts (δ) are in parts per million. Thin-layer chromatography (TLC) analysis was performed with Kieselgel 60 F₂₅₄ (Merck) plates and visualized using UV light.

Analytical HPLC/MS was performed on an Agilent HP1100 LC system using reverse phase Luna C18 columns (3 μm, 50×4.6 mm), gradient 5-95% B (A=water/0.1% Formic acid, B=acetonitrile/0.1% Formic acid) over 13.0 min, flow=1.25 mL/min. UV spectra were recorded at 220 and 254 nm using a G1315B DAD detector. Mass spectra were obtained over the range m/z 150 to 800 on a LC/MSD SL G1956B detector. Data were integrated and reported using ChemStation and ChemStation Data Browser softwares.

### Intermediates

The intermediates for the preparation of the examples described herein are shown below (FIG. 1):

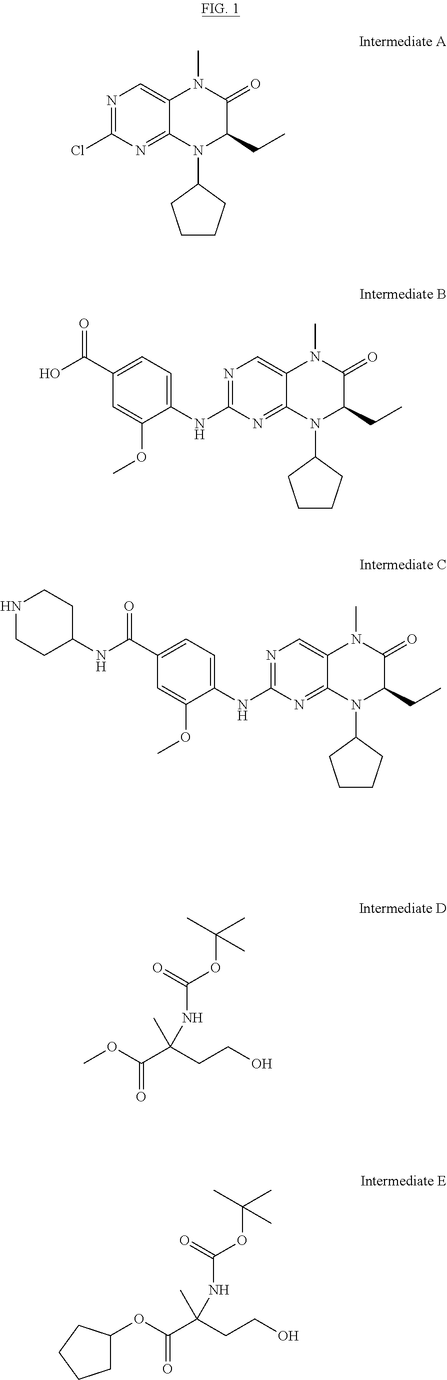

### Intermediate A:

### (7R)-2-Chloro-8-cyclopentyl-7-ethyl-5-methyl-7,8-dihydropteridin-6(5H)-one

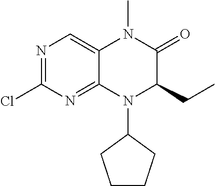

The title compound was prepared using methodology described in WO2004076454.

### Intermediate B:

### 4-{[(7R)-8-Cyclopentyl-7-ethyl-5-methyl-6-oxo-5,6,7,8-tetrahydropteridin-2-yl]amino}-3-methoxybenzoic acid

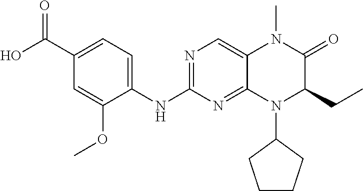

The title compound was prepared using methodology described in WO2004076454.

### Intermediate C:

### 4-{[(7R)-8-Cyclopentyl-7-ethyl-5-methyl-6-oxo-5,6,7,8-tetrahydropteridin-2-yl]amino}-3-methoxy-N-piperidin-4-ylbenzamide

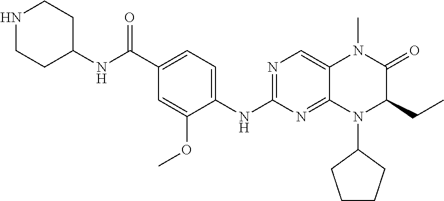

The title compound was prepared from Intermediate B by the following methodology:

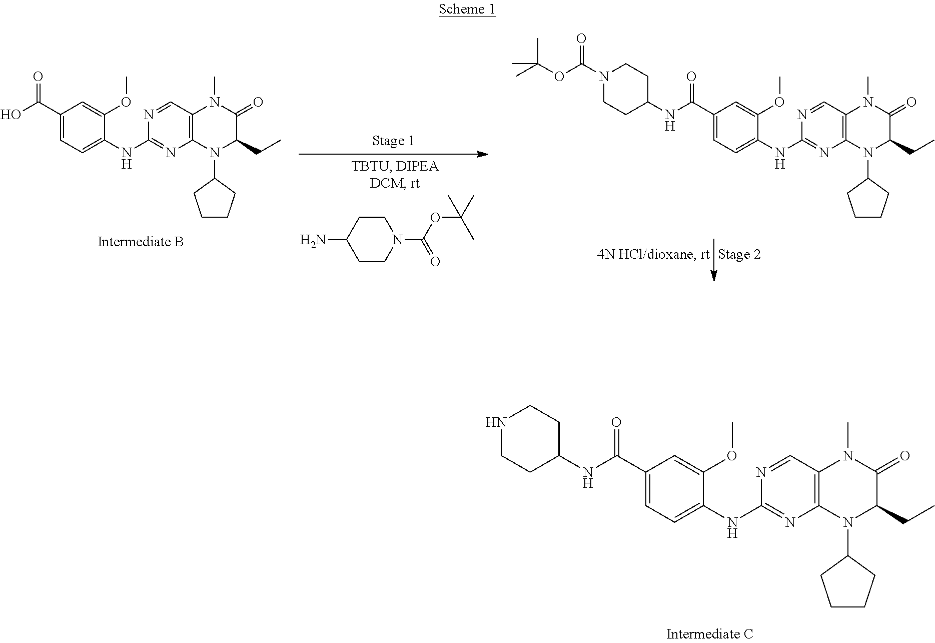

### Stage 1—tert-butyl 4-[(4-{[(7R)-8-cyclopentyl-7-ethyl-5-methyl-6-oxo-5,6,7,8-tetrahydro pteridin-2-yl]amino}-3-methoxybenzoyl)amino]piperidine-1-carboxylate

To a suspension of Intermediate B (500 mg, 1.18 mmol) in DCM (20 mL) was added TBTU (415 mg, 1.29 mmol) and DIPEA (0.41 mL, 2.35 mmol). The reaction mixture was stirred at RT for 30 min and tert-butyl 4-aminopiperidine-1-carboxylate (282 mg, 1.41 mmol) was added. The reaction mixture was stirred at RT for 30 min, diluted with DCM (30 mL), washed with water (2×30 mL), dried (MgSO₄), and concentrated under reduced pressure to leave a thick brown oil. Trituration with Et₂O/heptane (1:3) afforded the title product as a beige solid (528 mg, 74%). ESMS m/z: 608 [M+H]⁺.

### Stage 2—4-{[(7R)-8-Cyclopentyl-7-ethyl-5-methyl-6-oxo-5,6,7,8-tetrahydropteridin-2-yl]amino}-3-methoxy-N-piperidin-4-ylbenzamide

Stage 1 product (528 mg, 0.87 mmol) was suspended in a 4N HCl in dioxane (10 mL) and the reaction mixture was stirred at RT for 1 hour and then concentrated under reduced pressure. The residue was triturated with Et₂O and then partitioned between DCM (100 mL) and sat Na₂CO₃ (50 mL). The organic layer was separated, washed with sat Na₂CO₃ (50 mL), dried (MgSO₄) and concentrated under reduced pressure to afford the title intermediate as a thick yellow oil, which solidified on standing (407 mg, 92%). ESMS m/z 508 [M+H]⁺. ¹H NMR (300 MHz, CDCl₃) δ: 8.56 (1H, dd, J=8.4, 3.5 Hz), 7.57-7.76 (2H, m), 7.39-7.44 (1H, m), 4.53 (1H, br.s.), 4.08-4.34 (2H, m), 3.98 (3H, d, J=4.7 Hz), 3.39-3.65 (2H, m), 3.29-3.38 (3H, m), 2.81-3.15 (2H, m), 1.41-2.44 (14H, m), 0.75-0.97 (3H, m).

### Intermediate D:

### Methyl N-(tert-butoxycarbonyl)-4-hydroxyisovalinate

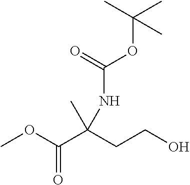

The title compound was prepared by the following methodology:

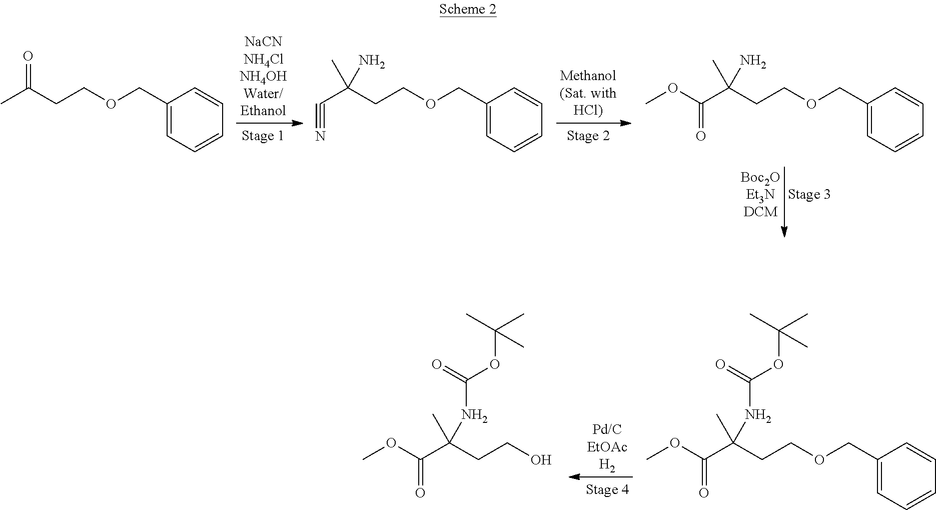

### Stage 1—2-amino-4-(benzyloxy)-2-methylbutanenitrile

A 2-neck round-bottomed flask (100 mL) was attached via an adaptor and rubber tubing to a funnel submerged in bleach. To this round-bottomed flask was added 98% sodium cyanide (1.81 g, 36.9 mmol) in water (3 mL), followed by ammonium chloride (2.17 g, 40.6 mmol) in lukewarm water (5 mL). Ammonium hydroxide (2.88 mL, 73.8 mmol) was then added, followed by 4-benzyloxy-2-butanone (6.58 g, 36.9 mmol) in ethanol (11 mL).

The resulting mixture was stirred at RT for 15 min, then at 60° C. for 2 hours. After cooling to RT the reaction mixture was concentrated under reduced pressure and partitioned between water (100 mL) and DCM (100 mL). The aqueous layer was extracted with DCM (3×75 mL). The combined organic layers were dried (MgSO₄), filtered and concentrated under reduced pressure. Purification on a 12 g silica column using a CombiFlash® Companion® (Teledyne Isco Inc) (EtOAc in heptane) gave the title product as a colourless oil (4.70 g, 62%). ESMS m/z: 205 [M+H]⁺; ¹H NMR (300 MHz, CDCl₃) δ: 7.36 (2H, br. s.), 7.33-7.25 (3H, m), 4.56-4.49 (2H, m), 3.73 (2H, t, J=6.1 Hz), 2.67-2.27 (2H, m), 1.49 (3H, s).

### Stage 2—Methyl 4-(benzyloxy)isovalinate

A 3-neck round-bottomed flask (500 mL) was attached via an adaptor and rubber tubing to a funnel submerged in saturated NaHCO₃. The round-bottomed flask was cooled to 0° C. with an ice bath and anhydrous methanol (50 mL) was added. The methanol was saturated with HCl (g) for 5 min. Stage 1 product (2.0 g, 9.8 mmol) in methanol (7 mL) was added to the reaction mixture and stirred at 65° C. for 48 hours. The reaction mixture was cooled to RT, concentrated under reduced pressure and partitioned between water (50 mL) and EtOAc (50 mL). The aqueous layer was isolated, saturated NaHCO₃ added and the product extracted into EtOAc (3×50 mL). The combined organic layers were dried (MgSO₄), filtered and concentrated under reduced pressure to afford the crude product as a dark yellow oil (2.32 g, 100%). ESMS m/z: 238 [M+H]⁺; ¹H NMR (300 MHz, CDCl₃) δ: 7.35-7.26 (5H, m), 5.39 (2H, br. s.), 4.57 (2H, d, J=5.8 Hz), 3.64 (3H, s), 3.61-3.52 (2H, m), 2.28-2.14 (2H, m), 1.53 (3H, s).

### Stage 3—Methyl 4-(benzyloxy)-N-(tert-butoxycarbonyl)isovalinate

To a solution of stage 2 product (2.32 g, 9.78 mmol) in DCM (20 mL) was added Et₃N (6.8 mL, 48.9 mmol). The reaction mixture was cooled to 0° C. in an ice bath and to it was added Boc₂O (2.56 g, 11.7 mmol) in 2 portions. The reaction was stirred at RT for 24 hours and the product was extracted onto silica under reduced pressure. Purification on a 12 g silica column using a CombiFlash® Companion® (Teledyne Isco Inc) (EtOAc in heptane) gave the title compound as a colourless oil (0.37 g, 15%). ESMS m/z: 360 [M+Na]⁺; ¹H NMR (300 MHz, CDCl₃) δ: 7.46-7.18 (5H, m), 6.03 (1H, br. s.), 4.45 (2H, m), 3.64 (3H, s), 3.55 (2H, t, J=5.6 Hz), 2.41-2.07 (2H, m), 1.58 (3H, s), 1.44 (9H, s).

### Stage 4—Methyl N-(tert-butoxycarbonyl)-4-hydroxyisovalinate

To a solution of stage 3 product (0.370 g, 1.10 mmol) in EtOAc (15 mL) was added 10% Pd/C (0.074 g, 20% w.w.). The system was evacuated and put under a hydrogen atmosphere (using a 3-way tap apparatus and hydrogen-filled balloon), this was repeated twice and the mixture was allowed to stir for 24 h at RT under a hydrogen atmosphere. The system was evacuated of hydrogen and the palladium residues filtered over Celite. The Celite was washed thoroughly with ethyl acetate and the filtrate solvent removed under reduced pressure. The crude residue was purified by column chromatography (50% EtOAc in heptane) to afford the title compound as a colourless oil (0.251 g, 93%). ESMS m/z: 270 [M+Na]⁺; ¹H NMR (300 MHz, CDCl₃) δ: 5.84 (1H, br. s.), 3.80-3.64 (5H, s), 2.30-2.07 (2H, m), 1.58 (3H, s), 1.43 (9H, s).

### Intermediate E:

### Cyclopentyl N-(tert-butoxycarbonyl)-4-hydroxyisovalinate

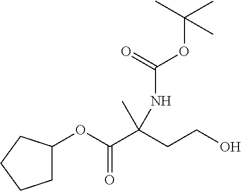

The title compound was prepared using the same methodology described for Intermediate D, replacing the methanol in stage 2 with cyclopentanol. ESMS m/z: 324 [M+Na]⁺; ¹H NMR (300 MHz, CDCl₃) δ: 5.99-5.75 (1H, m), 5.15 (1H, t, J=5.6 Hz), 3.67 (2H, t, J=5.9 Hz), 2.17-1.92 (2H, m), 1.89-1.51 (8H, m), 1.49 (3H, s), 1.38 (9H, s).

### Example 1

### Methyl 4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}isovalinate

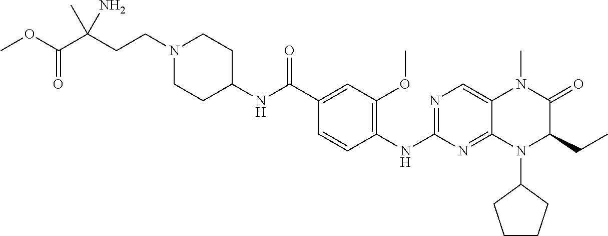

The title compound was prepared by the following methodology:

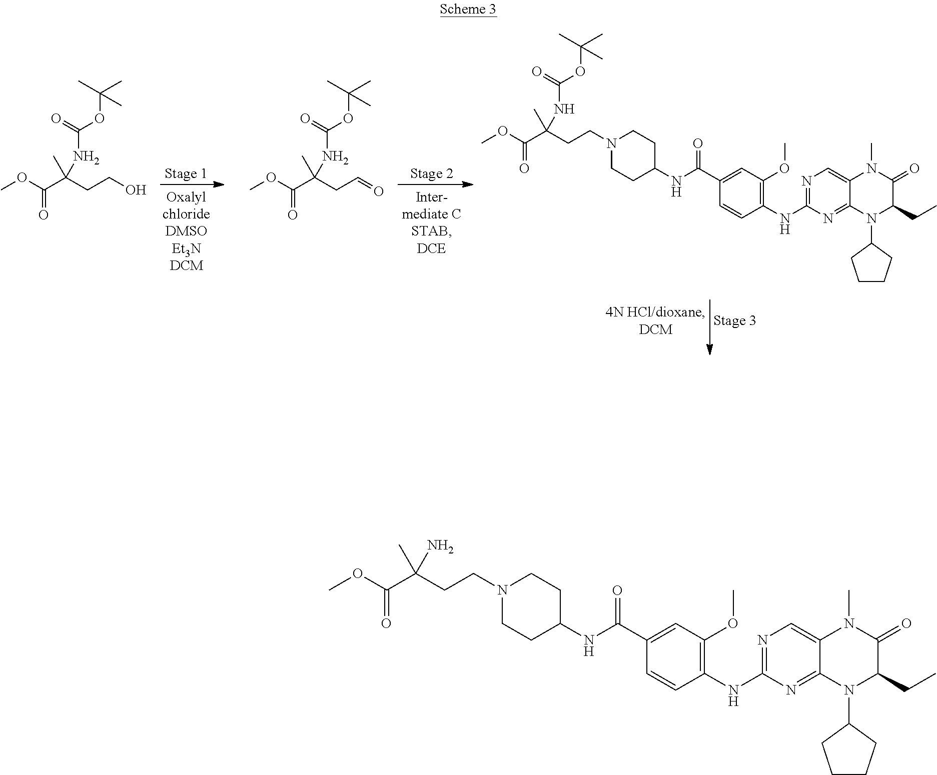

### Stage 1—Methyl 4-oxoisovalinate

A solution of DCM (5 mL) and oxalyl chloride (0.055 mL, 0.62 mmol) was stirred under a nitrogen atmosphere and cooled to an internal temperature of −70° C. DMSO (0.040 mL, 0.56 mmol) was added slowly, ensuring the temperature remained below −65° C. A solution of Intermediate D (0.068 g, 0.26 mmol) in DCM (2 mL) was added slowly, followed by Et₃N (0.310 mL, 2.20 mmol), keeping the temperature below −65° C. at all times. The reaction was then allowed to warm to RT and the solvent was removed under reduced pressure to afford the crude product as a pale yellow solid. This crude mixture was taken on to the reductive amination step without further purification.

### Stage 2—Methyl N-(tert-butoxycarbonyl)-4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}isovalinate

A solution of Intermediate C (0.131 g, 0.26 mmol) and stage 1 product (0.064 g, 0.26 mmol) in DCE (3.5 mL) was stirred at RT for 15 min. Sodium triacetoxyborohydride (0.165 g, 0.78 mmol) was added and the reaction mixture stirred at RT overnight. Saturated NaHCO₃ (10 mL) and DCM (20 mL) were added and the aqueous layer extracted with DCM (3×15 mL). The combined organic layers were dried (MgSO₄), filtered and concentrated under reduced pressure to afford the crude product as a yellow oil (0.210 g). ESMS m/z: 737 [M+H]⁺. ¹H NMR (300 MHz, MeOD), 7.90 (1H, d, J=8.4 Hz), 7.64 (2H, s), 7.59 (1H, d J=9.8 Hz), 4.52 (1H, dd, J=3.2, 6.4 Hz), 4.33 (1H, t J=8.5 Hz), 4.30-4.18 (1H, m), 4.00 (3H, m), 3.93 (3H, s), 3.76-3.69 (2H, m), 3.33 (3H, s), 3.27-3.15 (4H, m), 2.46 (2H, t, J=8.4 Hz), 2.28 (2H, d J=12.8 Hz), 2.08-1.90 (8H, m), 1.67-1.56 (7H, m), 1.38 (9H, s), 0.88 (3H, t, J=7.4 Hz).

### Stage 3—Methyl 4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}isovalinate

To a solution of stage 2 product (0.030 g, 0.04 mmol) in DCM (1 mL) was added 4N HCl in dioxane (3 mL). The reaction was stirred at RT for 2 hours and the solvent was removed under reduced pressure. The resulting residue was taken up in a 1:1 ratio of acetonitrile and water (1.3 mL) and purified by preparative HPLC. The pure fractions were combined and dried on the freeze-drier to afford the title compound as a white solid. (0.023 g, 12%). ESMS m/z: 637 [M+H]⁺; ¹H NMR (300 MHz, MeOD), 7.90 (1H, d, J=8.4 Hz), 7.64 (2H, s), 7.59 (1H, d J=9.8 Hz), 4.52 (1H, dd, J=3.2, 6.4 Hz), 4.33 (1H, t J=8.5 Hz), 4.30-4.18 (1H, m), 4.00 (3H, m), 3.93 (3H, s), 3.76-3.69 (2H, m), 3.33 (3H, s), 3.27-3.15 (4H, m), 2.46 (2H, t, J=8.4 Hz), 2.28 (2H, d J=12.8 Hz), 2.08-1.90 (8H, m), 1.67-1.56 (7H, m), 0.88 (3H, t, J=7.4 Hz).

### Example 2

### Cyclopentyl 4-{4-[(4-{[(7R)-8-cyclopentyl-7-ethyl-5-methyl-6-oxo-5,6,7,8-tetrahydro pteridin-2-yl]amino}-3-methoxybenzoyl)amino]piperidin-1-yl}isovalinate

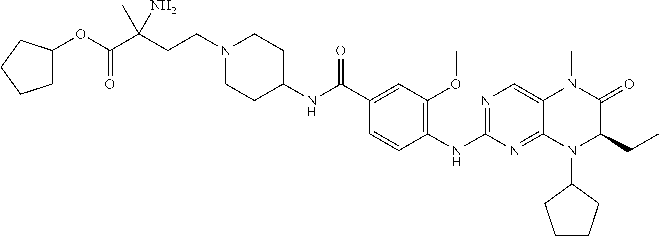

This compound was prepared from Intermediate E using the same methodology described for Example 1. ESMS m/z: 691 [M+H]⁺; ¹H NMR (300 MHz, MeOD), 7.89 (1H, d, J=8.3 Hz), 7.63 (2H, s), 7.58 (1H, dd, J=8.4, 1.6 Hz), 5.35 (1H, t, J=5.6 Hz), 4.51 (1H, dd, J=6.4, 3.0 Hz), 4.39-4.12 (2H, m), 3.99 (3H, s), 3.71 (2H, br.s.), 3.33 (3H, br.s.), 3.29-3.07 (4H, m), 2.43 (2H, t, J=8.5 Hz), 2.27 (2H, d, J=14.3 Hz), 2.16-1.66 (20H, m), 1.64 (3H, s), 0.87 (3H, t, J=7.4 Hz).

### Example 3

### 4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}isovaline

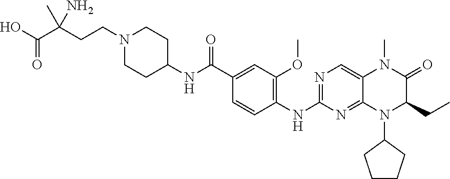

The title compound was prepared by the following methodology:

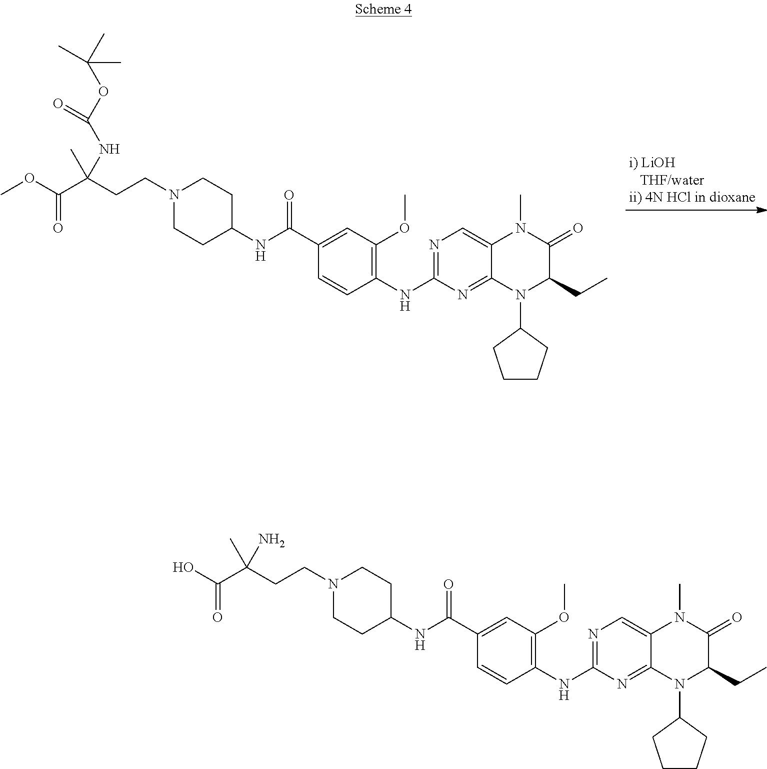

To a solution of Example 1 stage 2 product (0.150 g, 0.20 mmol) in THF (5 mL) and water (5 mL) was added LiOH (0.098 g, 4.08 mmol). The reaction mixture was stirred at 40° C. overnight. THF was removed under reduced pressure and the resulting solution was acidified to pH 1-2 with 2N HCl. The product was extracted into tert-butanol (3×30 mL) and concentrated to dryness under reduced pressure. The product was slurried in EtOAc and isolated by filtration to give 0.09 g of solid. 0.03 g was treated with 4N HCl in dioxane (1 mL) for 1 hour. Solvent was removed under reduced pressure, product taken in 1:1 ratio of AcCN/H₂O (1.3 mL) and purified by preparative HPLC. The pure fractions were combined and dried on the freeze-drier to afford the title compound as a white solid. (0.010 g, 7.9%). ESMS m/z: 623 [M+H]⁺; ¹H NMR (300 MHz, MeOD), 7.95 (1H, d, J=8.3 Hz), 7.64 (2H, s), 7.58 (1H, dd J=1.8, 8.3 Hz), 4.51 (1H, dd, J=4.0, 6.6 Hz), 4.35 (1H, t J=9.1 Hz), 4.27-4.18 (1H, m), 4.00 (3H, m), 3.72-3.663 (2H, m), 3.33 (3H, s), 3.27-3.15 (4H, m), 2.46-2.41 (2H, m), 2.30-2.24 (2H, m), 2.10-1.88 (8H, m), 1.70-1.63 (7H, m), 0.88 (3H, t, J=7.4 Hz).

### Measurement of Biological Activity

### PLK1 Enzyme Assay

The ability of compounds to inhibit PLK-1 kinase activity was measured in an assay performed by Invitrogen (Paisley, UK). The Z′-LYTE™ biochemical assay employs a fluorescence-based, coupled-enzyme format and is based on the differential sensitivity of phosphorylated and non-phosphorylated peptides to proteolytic cleavage. The peptide substrate is labelled with two fluorophores—one at each end—that make up a FRET pair. In the primary reaction, the kinase transfers the gamma-phosphate of ATP to a single serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e., coumarin) and acceptor (i.e., fluorescein) fluorophores on the FRET-peptide, whereas uncleaved, phosphorylated FRET-peptides maintain FRET. A radiometric method, which calculates the ratio (the Emission Ratio) of donor emission to acceptor emission after excitation of the donor fluorophore at 400 nm, is used to quantitate reaction progress.

The final 10 μL Kinase Reaction consists of 2.8-25.3 ng PLK1, 2 μM Ser/Thr 16 Peptide substrate and ATP in 50 mM HEPES pH 7.5, 0.01% BRIJ-35, 10 mM MgCl2, 1 mM EGTA. The assay is performed at an ATP concentration at, or close to, the Km. After the 60 minute Kinase Reaction incubation at RT, 5 μL of a 1:8 dilution of Development Reagent is added. The assay plate is incubated for a further 60 minutes at RT and read on a fluorescence plate reader.

Duplicate data points are generated from a ⅓ log dilution series of a stock solution of test compound in DMSO. Nine dilutions steps are made from a top concentration of 10 μM, and a ‘no compound’ blank is included. Data is collected and analysed using XLfit software from IDBS. The dose response curve is curve fitted to model number 205 (sigmoidal dose-response model). From the curve generated, the concentration giving 50% IC50 inhibition is determined and reported.

IC50 results were allocated to one of 3 ranges as follows:

### Range A: IC50<100 nM,

Range B: IC50 from 100 nM to 500 nM;

### and Range C: IC50>500 nM.

NT=Not tested

The results obtained for compounds of the Examples herein are shown below (Table 1).

### Cell Inhibition Assay

Cells were seeded in 96W tissue culture plates (1 well=30 mm²) in 50 μl of the appropriate culture medium (see below). Seeding densities were cell-line dependent: HCT-116=1000 cells per well, Hut-78=2250 cells per well, U937 cells=2000 cells per well. 24 hrs later 50 μL of the compound prepared in the same medium was added as 4 fold dilutions to give final concentrations in the range 0.15 nM-2500 nM (n=6 for each concentration). The plates were then incubated at 37° C., 5% CO₂ for 72 hrs. Cell proliferation was assessed using WST-1 (a metabolic indicator dye, Roche Cat no. 1 644 807) according to the manufacturers instructions. The results were calculated as percentage of vehicle response and IC50 values represent the concentration of compound that inhibited the vehicle response by 50%.

HCT-116 Culture Medium—Dulbeccos MEM (Sigma D6546) plus 10% heat inactivated fetal calf serum (Hyclone SH30071 Thermo Fischer Scientific) containing 2 mM Glutamine (Sigma cat no G-7513) and 50 U/mL Penicillin and Streptomycin Sulphate (Sigma Cat no P-0781).

Hut-78 & U937 culture media: RPMI1640 (Sigma R0883) plus 10% heat inactivated fetal calf serum, as above and supplemented with 2 mM Glutamine and 50 U/mL Penicillin and Streptomycin Sulphate (details as above).

IC50 results were allocated to one of 3 ranges as follows:

### Range A: IC50<100 nM,

Range B: IC50 from 100 nM to 500 nM;

### and Range C: IC50>500 nM.

NT=Not tested

The results obtained for compounds of the Examples herein are shown below (Table 1).

TABLE 1

| | | Inhibitor | | |
|---|---|---|---|---|
| | Inhibitor | Activity vs | Inhibitor | Inhibitor |
| Example | Activity vs | HCT 116 | Activity vs | Activity vs |
| Number | PLK1 | cell line | Hut-78 cell line | U937 cell line |
| 1 | A | B | A | A |
| 2 | A | A | A | A |
| 3 | A | NT | NT | NT |

### Broken Cell Carboxylesterase Assay

Any given compound of the present invention wherein R₇ is an ester group, may be tested to determine whether it meets the requirement that it be hydrolysed by intracellular esterases, by testing in the following assay.

### Preparation of Cell Extract

U937 or HCT 116 tumour cells (˜10⁹) were washed in 4 volumes of Dulbeccos PBS (˜1 litre) and pelleted at 525 g for 10 min at 4° C. This was repeated twice and the final cell pellet was resuspended in 35 mL of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 mL) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at RT by LCMS (Sciex API 3000, HP1100 binary pump, CTC PAL). Chromatography was based on an AceCN (75×2.1 mm) column and a mobile phase of 5-95% acetonitrile in water/0.1% formic acid.

The table below (Table 2) presents data showing that several amino acid ester motifs, conjugated to various intracellular enzyme inhibitors by several different linker chemistries are all hydrolysed by intracellular carboxyesterases to the corresponding acid.

TABLE 2

| | | | Hydroly- | |
|---|---|---|---|---|
| | | | sis Rate | |
| | | | Range | |
| | | | U937- | Preparation |
| | | | Cells | of |
| | | | ( pg/mL/ | amino ester |
| Structure of amino acid ester conjugate | R | Linker | min) | conjugate |
| 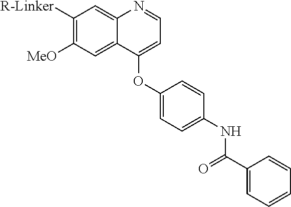 | 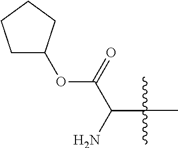 | —CH2CH2O— | 100-1000 | WO2006117552 |
| 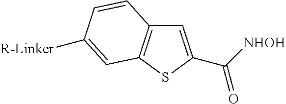 | 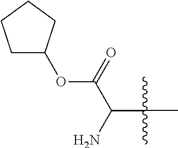 | 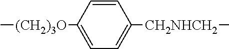 | 1000- 50000 | WO2006117548 |
| 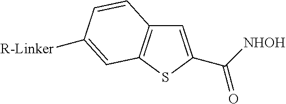 | 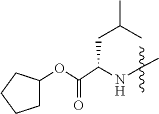 | 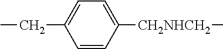 | >50000 | WO2006117549 |
| 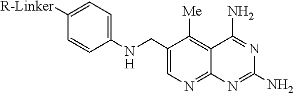 | 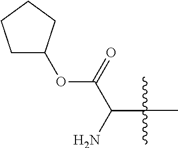 | —CH2CH2O— | >50000 | WO2006117567 |
| 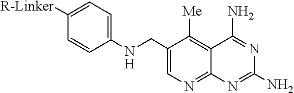 | 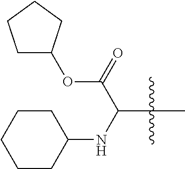 | —CH2CH2O— | 1000- 50000 | WO2006117567 |
| 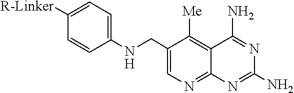 | 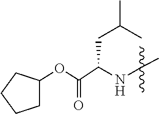 | —CH2— | 1000- 50000 | WO2006117567 |
| 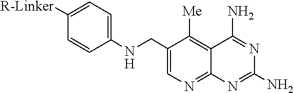 | 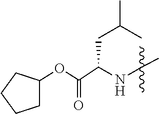 | —CO— | >50000 | WO2006117567 |
| 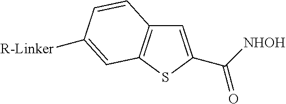 | 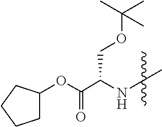 | 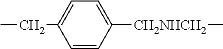 | >50000 | WO2006117549 |
| 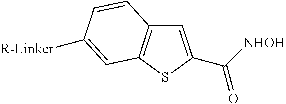 | 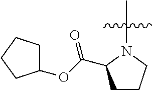 | 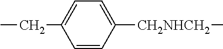 | >50000 | WO2006117549 |

1. A compound of formula (I), or a salt thereof: 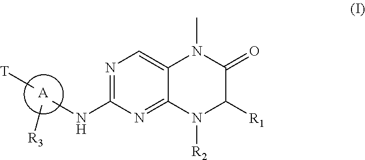
wherein
R₁ is hydrogen, or an optionally substituted (C₁-C₆)alkyl, (C₂-C₆)alkenyl, (O₂—C₆)alkynyl or (C₃-C₆)cycloalkyl group;
R₂ is hydrogen, or an optionally substituted (C₁-C₆)alkyl, (C₂-C₆)alkenyl, (O₂—C₆)alkynyl or (C₃-C₆)cycloalkyl group;
R₃ is hydrogen, —CN, hydroxyl, halogen, optionally substituted (C₁-C₆)alkyl, (O₂—C₆)alkenyl, (C₂-C₆)alkynyl or (C₃-C₆)cycloalkyl, —NR₅R₆ or C₁-C₄ alkoxy, wherein R₅ and R₆ are independently hydrogen or optionally substituted (C₁-C₆)alkyl;
ring A is an optionally substituted mono- or bi-cyclic carbocyclic or heterocyclic ring or a ring system having up to 12 ring atoms;
T is a radical of formula R-L¹-Y¹— wherein
Y¹ is a bond, —O—, —S—, —NR₆—, —(C═O)—, —S(O₂)—, —(C═O)NR₆—, —NR₆(C═O)—, —S(O₂)NR₆—, —NR₆S(O₂)—, or —NR₆(C═O)NR₉—, wherein R₆ and R₉ are independently hydrogen or optionally substituted (C₁-C₆)alkyl;
L¹ is a divalent radical of formula -(Alk¹)ₘ(Q)ₙ(Alk²)ₚ- wherein
m, n and p are independently 0 or 1,
Q is (i) an optionally substituted divalent mono- or bicyclic carbocyclic or heterocyclic radical having 5-13 ring members, or (ii), in the case where p is 0, a divalent radical of formula -Q¹-X²— wherein X² is —O—, —S— or NR^{A}— wherein
R^{A} is hydrogen or optionally substituted C₁-C₃ alkyl, and Q¹ is an optionally substituted divalent mono- or bicyclic carbocyclic or heterocyclic radical having 5-13 ring members,
Alk¹ and Alk² independently represent optionally substituted divalent (C₃-C₆)cycloalkyl radicals, or optionally substituted straight or branched, (C₁-C₆)alkylene, (C₂-C₆)alkenylene, or (C₂-C₆)alkynylene radicals which may optionally contain or terminate in an ether (—O—), thioether (—S—) or amino (—NR^{A}—) link wherein R^{A} is hydrogen or optionally substituted (C₁-C₃)alkyl;
R is a radical of formula (X) 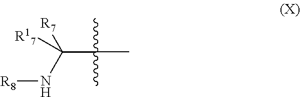
wherein
R₇ is a carboxylic acid group (—COOH), or an ester group which is hydrolysable by one or more intracellular carboxylesterase enzymes to a carboxylic acid group;
R¹₇ is the side chain of a natural or non-natural alpha-amino acid, in which any functional groups are protected, but R¹₇ is not hydrogen;
R₈ is hydrogen; or optionally substituted C₁-C₆ alkyl, C₃-C₇ cycloalkyl, aryl or heteroaryl or —(C═O)R₆, —(C═O)OR₆, or —(C═O)NR₆ wherein R₆ is hydrogen or optionally substituted (C₁-C₆)alkyl;

2. A compound as claimed in claim 1 wherein R₁ is ethyl.

3. A compound as claimed in claim 1 wherein R₂ is cyclopentyl.

4. A compound as claimed in claim 1 wherein ring A is an optionally substituted phenyl ring.

5. A compound as claimed in claim 1 wherein R₇ is of formula —(C═O)OR₁₀ wherein R₁₀ is R₁₁R₁₂R₁₃C— wherein
(i) R₁₁ is hydrogen, fluorine or optionally substituted (C₁-C₃)alkyl-(Z¹)ₐ—[(C₁-C₃)alkyl]_{b}- or (C₂-C₃)alkenyl-(Z¹)ₐ—[(C₁-C₃)alkyl]_{b}- wherein a and b are independently 0 or 1 and Z¹ is —O—, —S—, or —NR₁₄— wherein R₁₄ is hydrogen or (C₁-C₃)alkyl; and R₁₂ and R₁₃ are independently hydrogen or (C₁-C₃)alkyl-;
(ii) R₁₁ is hydrogen or optionally substituted R₁₅R₁₆N—(C₁-C₃)alkyl- wherein R₁₅ is hydrogen or (C₁-C₃)alkyl and R₁₆ is hydrogen or (C₁-C₃)alkyl; or R₁₅ and R₁₆ together with the nitrogen to which they are attached form an optionally substituted monocyclic heterocyclic ring of 5- or 6-ring atoms or bicyclic heterocyclic ring system of 8 to 10 ring atoms, and R₁₂ and R₁₃ are independently hydrogen or (C₁-C₃)alkyl-; or
(iii) R₁₁ and R₁₂ taken together with the carbon to which they are attached form an optionally substituted monocyclic carbocyclic ring of from 3 to 7 ring atoms or bicyclic carbocyclic ring system of 8 to 10 ring atoms, and R₁₃ is hydrogen.
and wherein in cases (i), (ii) and (iii) above, “alkyl” includes fluoroalkyl.

6. A compound as claimed in claim 5 wherein R₁₀ is methyl, trifluoromethyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl, or morpholinoethyl.

7. A compound as claimed in claim 5 wherein R₁₀ is cyclopentyl.

8. A compound as claimed in claim 1 wherein R¹₇ is phenyl, or heteroaryl such as pyridyl, or a group of formula —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ, and R_{b} are each independently (C₁-C₆)alkyl, (C₂-C₆)alkenyl, (C₂-C₆)alkynyl, phenyl(C₁-C₆)alkyl, or a group as defined for Rₐ, below other than hydrogen, or
Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

9. A compound as claimed in claim 1 wherein R¹₇ is H-Alk⁴-, phenyl, monocyclic heterocyclyl, C₃-C₇ cycloalkyl, phenyl(Alk⁴)-, heterocyclyl(Alk⁴)-, or C₃-C₇ cycloalkyl(Alk⁴)-, wherein the heterocyclyl part is monocyclic heterocyclyl having 3-7 ring atoms, and wherein -Alk⁴- is a straight or branched, divalent (C₁-C₆)alkylene, (C₂-C₆)alkenylene, or (C₂-C₆)alkynylene radical which may optionally be interrupted by, or terminate in, an ether (—O—), thioether (—S—) or amino (—NR^{A}—) link wherein R^{A} is hydrogen or optionally substituted (C₁-C₃)alkyl, and wherein the Alk⁴-, or cyclic part is optionally substituted.

10. A compound as claimed in claim 1 wherein R¹₇ is methyl, ethyl, n- or iso-propyl, or n-, sec- or tert-butyl.

11. A compound as claimed in claim 1 wherein R¹₇ is methyl.

12. A compound as claimed in claim 1 wherein R₈ is hydrogen.

13. A compound as claimed in claim 1 wherein L¹ is
—CH₂—, —CH₂CH₂— or —CH₂CH₂CH₂—, and Y¹ is —NHC(═O)—.

14. A compound as claimed in claim 1 which is the subject of any of the Examples herein.

15. A pharmaceutical composition comprising a compound as claimed in claim 1, together with a pharmaceutically acceptable carrier.

16. A composition comprising a compound as claimed in claim 1 in an amount for inhibition of PLK1 activity in vitro or in vivo.

17. A method of treatment of conditions mediated by PLK1 activity, which comprises administering to a subject suffering such disease an effective amount of a compound of formula (I) as claimed in claim 1.

18. The method as claimed in claim 17 for treatment of cell proliferative diseases

19. The method as claimed in claim 17 for treatment of cancer.
